(19) United States
(12) Patent Application Publication
(10) Pub. No.: US 2006/0129192 A1
(43) Pub. Date: Jun. 15, 2006

(54) High-energy battery power source for implantable medical use

(76) Inventors: Wilson Greatbatch, Williamsville, NY (US); Jeffrey Deal, Clarence, NY (US); Glenn Thomas, East Amherst, NY (US)

Correspondence Address: WALTER W. DUFT;LAW OFFICES OF WALTER W. DUFT, 8616 MAIN ST, SUITE 2, WILLIAMSVILLE, NY, 14221, US

(21) Appl. No.: 11/274,926

(22) Filed: Nov. 15, 2005

Related U.S. Application Data
(63) Continuation-in-part of application No. 10/994,565, filed on Nov. 22, 2004.

(51) Int. Cl. A61N 1/39

(52) U.S. Cl. 607/5

(57) ABSTRACT

A high energy battery power source suitable for use in an implantable medical device includes an input, an output, and two or more battery modules each comprising two or more battery cells. The battery cells are of relatively low voltage and permanently configured within each battery module in an electrically parallel arrangement in order to provide a desired current discharge level needed to achieve high-energy output. A switching system configures the battery modules between a first configuration wherein the battery modules are electrically connected in parallel to each other and to the input in order to receive charging energy at the relatively low voltage, and a second configuration wherein the battery modules are electrically connected in series to each other in order to provide to the output a relatively high voltage corresponding to the number of battery modules at a current level corresponding to the number of battery cells in a single battery module. An alternate embodiment permanently connects the battery modules in series so that no switching system is need for discharging and charging. A technique that provides for the control of discharge voltages on a pulse-to-pulse basis is also disclosed.

### CROSS-REFERENCE TO RELATED APPLICATIONS

This application is a continuation-in-part of U.S. patent application Ser. No. 10/994,565, filed on Nov. 22, 2004 and entitled “High Energy Battery Power Source For Implantable Medical Use.”

### BACKGROUND OF THE INVENTION

1. Field of the Invention

The present invention relates to implantable defibrillators, ICDs (Implantable Cardioverter-Defibrillators) and other battery powered medical devices designed to provide high-energy electrical stimulation of living tissue for therapeutic purposes.

2. Description of Prior Art

High-energy battery powered medical devices designed for implantable use, such as implantable defibrillators and ICDs, are designed to deliver a strong electrical shock to the heart when called upon to correct an onset of tachyarrhythmia. In traditional devices of this type, the high-energy pulse is produced by charging one or more high-voltage energy storage capacitors from a low voltage battery and then rapidly discharging the capacitors to deliver the intended therapy. This concept is widely practiced and disclosed in numerous patents, including U.S. Pat. No. 4,475,551 of Mirowski dated Oct. 9, 1984. Additionally, much clinical data on defibrillation therapy has been collected and published. See, for example, Gregory P. Walcott, et al. “Mechanisms of Defibrillation for Monophasic and Biphasic Waveforms.” Pacing and Clinical Electrophysiology. March 1994:478 and Andrea Natale, et al. “Comparison of Biphasic and Monophasic Pulses.” Pacing and Clinical Electrophysiology. July 1995:1354.

As an alternative to using high-energy capacitors for defibrillation of a patient via an implantable device, U.S. Pat. No. 5,369,351 of Adams (the “'351 patent”) proposes a high-voltage charge storage array based on batteries. The '351 patent specifically identifies a Lithium Vanadium-Oxide (LiV₆O₁₃) battery cell comprising a polymer electrolyte that can be manufactured in foil sheets of thickness less than 0.005 inches (127 μm). These cells are said to have an energy-storage capacity of over 1000 times that of capacitors of equivalent volume. Each cell produces a voltage output of approximately three volts and it is stated that an array of two hundred such cells connected in series will produce the 600 volts commonly delivered by capacitor-based defibrillators. In one exemplary construction, the array of two hundred cells is configured in four 50-cell blocks that would each deliver 150 volts when in series, for a total of 600 volts. To facilitate charging of these cell blocks using a low-voltage charge source, such as a conventional 3-4 volt primary battery, a plurality of switches are provided, one for each cell, so that the cells can be switched from an all-series configuration, as required for high-voltage discharge, to an all-parallel configuration, in which each cell of each cell block can be charged in parallel by the low voltage charge source.

Notwithstanding the asserted advantages of the battery-cell array of the '351 patent for delivering defibrillatory energy to living tissue, there are aspects of the proposed array that suggest it may not be entirely suited for implantable use. For instance, assuming a most efficient configuration in which the batteries cells are stacked on top of each other, the total thickness of a two-hundred cell array at 127 μm per cell would be 200×127=25,400 μm=2.54 cm=1 inch. This is substantially thicker than commercially available ICDs on the market today, which average around 2 cm in thickness. The '351 patent is also silent with respect to the discharge current capacity of the disclosed battery cells. The amount of energy conventionally delivered by an implantable ICD is about 30 joules. Delivery of this amount of energy is not only a function of the voltage, but also the discharge current. It is not clear whether the battery cells disclosed in the '351 patent would provide sufficient discharge current to generate the required energy if the cells are arranged in series as disclosed. Moreover, the maximum discharge current of polymer-electrolyte batteries is typically given as a function of cell cross-sectional area. There is no mention in the '351 patent of the cross-sectional dimensions of the disclosed battery cells, and no indication of whether cells with sufficient discharge current capability could be produced within the cross-sectional constraints of the power supply section of a conventional ICD. The '351 patent also fails to provide information regarding the self-discharge characteristics of the disclosed battery cells, which are important when determining recharge requirements. Lastly, the switching system of the '351 patent, in which a switch is provided for each battery cell (and with three switches per cell being provided in some embodiments) raises a question of how the circuit resistance introduced by the switches impacts the peak discharge current of the battery-cell array. The impact on overall system volume of having so many switches is another question left unanswered.

U.S. Pat. No. 6,782,290 of Schmidt (the “'290 patent”) is similarly deficient. The '290 patent is directed to an implantable medical device with a rechargeable thin-film microbattery battery power source. In the only disclosed example in which battery electrical characteristics are discussed, it is said that three 4-volt microbatteries can be configured in a parallel configuration for charging, and then reconfigured in a series configuration via device programming to create a 12-volt microbattery for discharge. This is far less than the voltage output required for an implantable defibrillator or ICD. Moreover, there is no discussion of current discharge requirements or how to achieve high energy levels as required for medical applications such as defibrillation.

It is to improvements in the practical design of high-energy implantable devices that the present invention is concerned. In particular, the invention is directed to a high-energy battery power source for use in an implantable defibrillator, ICD or other battery-powered medical device. Advantageously, the invention accomplishes the foregoing while adhering to commonly accepted constraints on size, shape and form factor.

### SUMMARY OF THE INVENTION

A high-energy power source according to exemplary embodiments of the invention comprises of a multiplicity of small-energy capacity rechargeable cells that are interconnected to provide a high-energy source suitable for delivering electrical stimulation therapy to living tissue. The power source includes an input, an output, and two or more battery modules each comprising two or more rechargeable battery cells. The battery cells are of relatively low voltage and permanently configured within each battery module in an electrically parallel arrangement in order to provide a desired current discharge level needed to achieve high-energy output. In a first embodiment, a switching system configures the battery modules between a first configuration wherein the battery modules are electrically connected in parallel to each other in order to receive charging energy from the input at the relatively low voltage, and a second configuration wherein the battery modules are electrically connected in series to each other in order to provide to the output a relatively high voltage corresponding to the number of battery modules at a current level corresponding to the number of battery cells in a single battery module. In a second embodiment, the battery modules are permanently arranged with a series electrical connection and recharged by one or more galvanically isolated outputs of a flyback converter circuit. Electrical connections may be provided at various points between battery modules to provide a range of fixed battery output voltages.

The power source can be conveniently formed using a stack of large surface area, thin-film battery cells, with the stack being sized to occupy the space of a conventional electrolytic capacitor as commonly used in implantable defibrillators and ICDs. The stack may include plural battery modules arranged one on top of the other. Within each battery module, the battery cells are also arranged on top of one another, preferably in a repeating pattern of electrolyte and electrode layers. Each module will thus be substantially free of insulation layers so as to minimize battery module thickness. All electrode layer sets associated with the cathode side of a battery module are interconnected, as are the electrode layer sets associated with the anode side of the battery module. This results in the battery cells of each battery module being connected in an electrically parallel arrangement.

### BRIEF DESCRIPTION OF THE DRAWINGS

The foregoing and other features and advantages of the invention will be apparent from the following more particular description of exemplary embodiments of the invention, as illustrated in the accompanying Drawings in which:

FIG. 1 is a diagrammatic plan view of an exemplary high-energy implantable medical device constructed in accordance with the principles of the present invention;

FIG. 2 is a diagrammatic cross-sectional view of a stack of battery modules, each of which comprises a stack of thin-film battery cells connected in parallel;

FIG. 3 is a detailed cross-sectional view showing a single exemplary battery cell that may be used in the battery modules of FIG. 2;

FIG. 4 is schematic diagram showing the battery module of FIG. 2 in combination with circuitry to provide a high-energy battery system subassembly with alternate charging and discharging circuits;

FIG. 5 is a schematic diagram showing multiple interconnected ones of the battery system subassembly of FIG. 4 to provide a high-energy, high-voltage battery system;

FIG. 6 is a simplified block diagram showing a primary battery, a high-energy, high-voltage battery system, a control system and a switching network for delivery of defibrillation energy according to one proposed circuit arrangement based on the principles of the invention;

FIG. 7 is a simplified block diagram showing an extra-corporeal charging system, a high-energy battery system, a control system and a switching network for delivery of defibrillation energy according to another proposed circuit arrangement based on the principles of the invention.

FIG. 8 is a schematic diagram showing four battery modules according to FIG. 2 permanently arranged with a series electrical connection to provide a high-energy, high-voltage battery system.

FIG. 9 is a simplified block diagram showing a primary battery and a flyback converter, a high-energy, high-voltage battery system, a control system and a switching network for delivery of defibrillation energy according to yet another proposed circuit arrangement based on the principles of the invention; and

FIG. 10 is a simplified block diagram showing a primary battery, a high-energy, high-voltage battery system, a control system and a switching network for delivery of defibrillation energy according to yet another proposed circuit arrangement based on the principles of the invention.

### DETAILED DESCRIPTION OF PREFERRED EMBODIMENTS

### INTRODUCTION

Exemplary high-energy battery power sources for use with implantable defibrillators, ICDs and other battery powered medical devices will now be described, together with an exemplary defibrillator that incorporates a high-energy battery power source therein. As indicated by way of summary above, the high-energy battery power source embodiments disclosed herein are characterized by a multiplicity of small capacity, thin-film rechargeable battery cells interconnected and densely packaged in a planar or rectilinear form factor. The rechargeable battery cells can be utilized on an intermittent basis to store and release electrical energy in order to deliver high-energy stimulus to living tissue for therapeutic purposes.

### Illustrated Embodiments

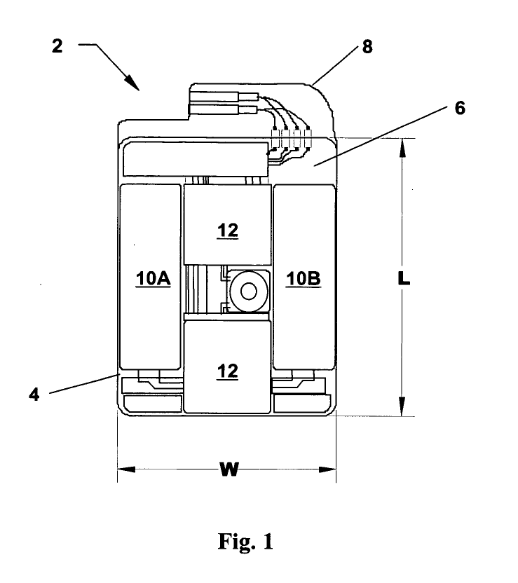

Turning now to the Drawings wherein like reference numerals signify like elements in all of the several views, FIG. 1 illustrates the physical construction and layout of an exemplary implantable device 2 designed to deliver high-energy stimulus to a patient using battery cells, and without the use of high-voltage energy storage capacitors. The device 2 is constructed with a casing 4 that defines a component cavity 6, and further includes a conventional connector block interface 8 situated at one end thereof. As can be seen, the device 2 has the usual shape, size and form factor of an implantable defibrillator or ICD. As such, the interior space available to house device components within the component cavity 6 will be on the order of 6.5 cm for the width dimension “W” and 8.0 cm for the length dimension “L.” Although not shown in FIG. 1, the interior height of the component cavity 6 (i.e., the dimension orthogonal to the page of FIG. 1) will be on the order of 1.7 cm. It will of course be appreciated that the foregoing dimensions are set forth by way of example only, and could no doubt be varied according to design needs. Evolution in standards and practices of the implantable device industry and medical community could also result in changes to the various dimensions of the device 2.

In FIG. 1, a pair of thin-film battery cell stacks 10A and 10B are situated along the sides of the component cavity 6 at locations where a pair of cylindrical electrolytic storage capacitors are often situated in a conventional defibrillator/ICD design. As such, each battery cell stack 10A and 10B can be approximately 2 cm wide by 6 cm in length. The height of each battery cell stack 10A and 10B must be within the interior height limit of the component cavity 6, i.e., on the order of 1.7 cm or less (or within whatever other cavity height dimension is present). Additional components 12 of the device 2, which are mostly conventional in nature (the only exception being certain battery-related circuit components to be described in more detail below), are situated between the battery cell stack 10A and 10B.

It should be understood that the number, size and location of cell stacks within an implantable device constructed in accordance with the invention could be varied from that shown in FIG. 1. For example, instead of two cell stacks, it may be feasible to use a single cell stack, perhaps situated at one end of the device housing and spanning the entire width of the component compartment. Other battery placement arrangements are disclosed in the '290 patent described by way of background above.

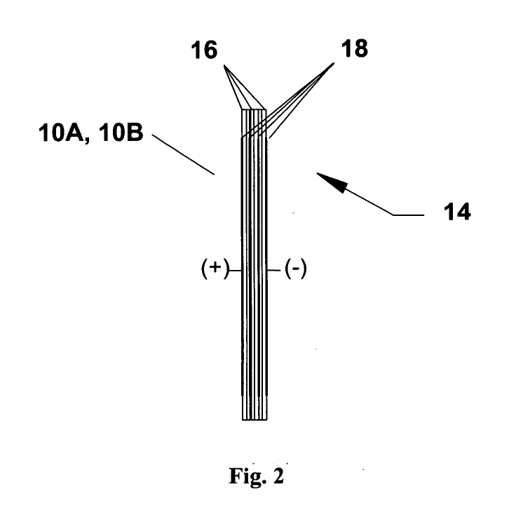

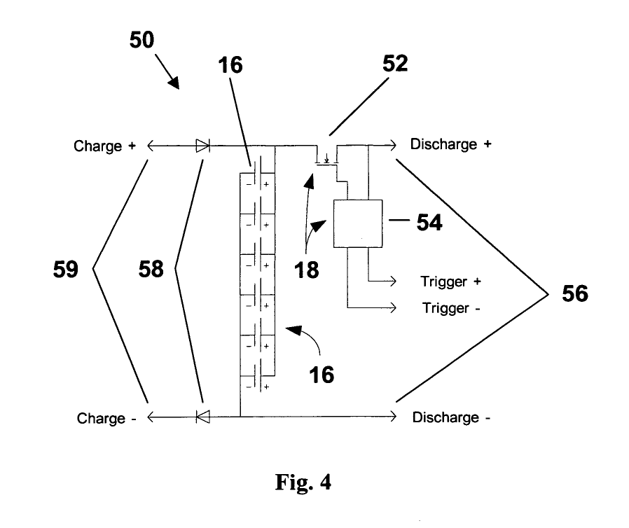

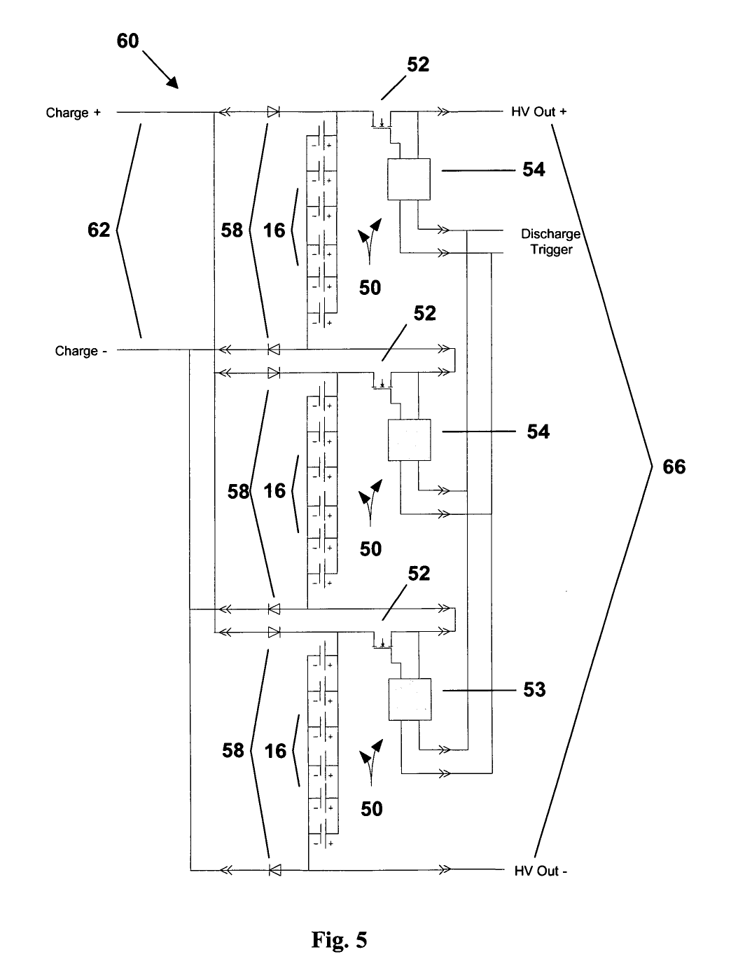

Turning now to FIG. 2, a representative battery cell stack configuration 14 is shown that can be used to form the battery cell stacks 10A and 10B. The cell stack 14 comprises several battery modules 16, each comprising plural thin-film battery cells that are hardwired in a parallel electrical configuration. The battery modules 16 are interconnected at 18 by way of switching circuitry to be described in more detail below with reference to FIGS. 4 and 5.

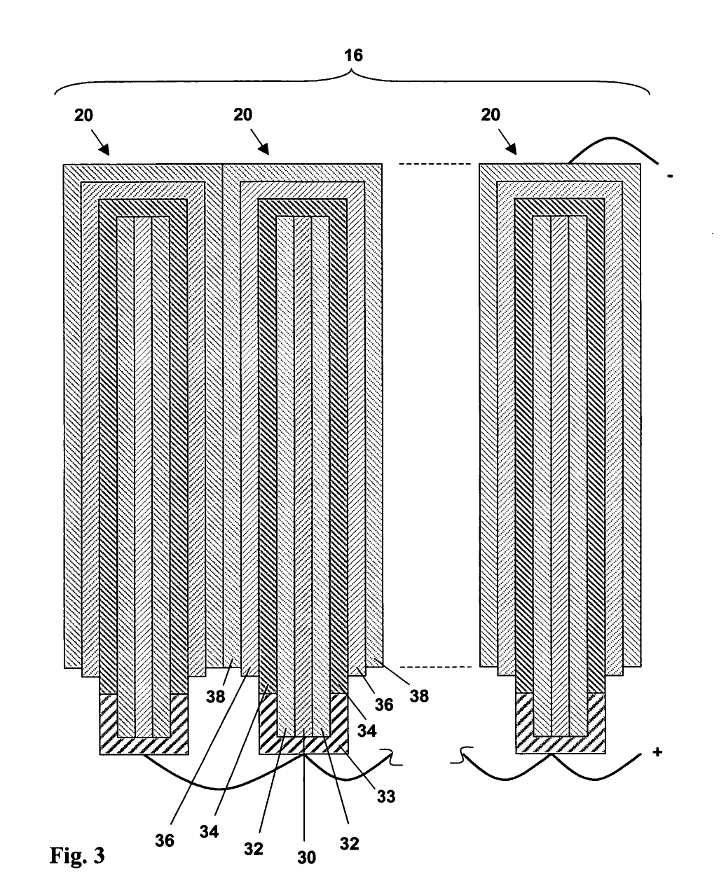

Turning now to FIG. 3, battery cells 20 that may be used in the battery module 16 are fabricated using thin-film cell construction techniques based on sputter deposition or equivalent means to deposit uniform patterned layers of high-purity materials. Such techniques are disclosed in U.S. Pat. No. 5,569,520 of Bates and U.S. Pat. No. 5,597,660 of Bates et al., the contents of which are incorporated herein by this reference. A specific thin-film battery cell design that may be used to construct the battery cells 20 is disclosed in U.S. Pat. No. 6,517,968 of Johnson et al. (the '968 patent). The contents of the '968 patent are incorporated herein by this reference. FIG. 4 of the '968 patent corresponds substantially to FIG. 3 herein. A similar design is disclosed in U.S. Published Patent Application No. 2004/0018424 of Zhang et al., the contents of which are also incorporated herein by this reference. The first-named inventor of the '698 patent is a co-inventor named in the '424 application.

As disclosed in the '968 patent, each battery cell 20 can be formed with a cathode current collector 30 made from a web of aluminum foil that is approximately 4 μm thick. Two cathodes 32 are respectively sputter-deposited on each side of the current collector 30 to a thickness of approximately 3 μm each. The cathodes 32 are made of a lithium intercalation compound, preferably a metal oxide such as LiNiO₂, V₂O₅, LiₓMn₂O₄, LiCoO₂, or TiS₂. A cathode current collector cap 33 made from aluminum or other compatible material can be applied over the exposed ends of the cathode current collector 30 and the cathodes 32.

Following deposition of the cathodes 32, the assembly is annealed at high temperature to crystallize the cathode material. The '968 patent instructs that this annealing of cathode material on a substrate such as the cathode current collector 30 results in a favorable orientation of cathode constituents that improves battery performance significantly in comparison to other thin-film battery constructions. Following the high-temperature treatment, electrolyte layers 34 are deposited on the cathodes 32 by sputtering of lithium orthophosphate, Li₅PO₄, in a nitrogen atmosphere to produce lithium phosphorous oxynitride coatings.

A pair of anodes 36 are then respectively applied to the electrolyte layers 34 by sputtering. The anodes 36 can be made of silicon-tin oxynitride, SiTON, or other suitable materials such as lithium metal, zinc nitride or tin nitride. Following deposition of the anodes 36, a pair of anode current collectors 38 are respectively deposited onto the anodes 36 by the sputtering of copper or nickel.

A critical element of each cell 20 is the electrolyte layer 34, which must be ionically conductive and non-reactive with the anode and cathode materials in order to provide a cell with stable lifetime properties. One example of a suitable electrolyte material is the above-mentioned lithium phosphorus oxynitride material (LiPON, LiₓPO_{y}N_{z}), which is disclosed and described in detail in the '968 patent, and in patents referenced therein. Unlike the electrolyte material found in the majority of primary and secondary cells that are currently commercially available, LiPON is a solid glassy compound which not only provides the physical separation between the anode and cathode layers but also exhibits excellent long term stability in contact with the reactive anode and cathode materials.

It should be understood that each individual cell 20 has a small surface area, perhaps 10 to 15 cm², with a total thickness of approximately 14 sum (see '968 patent). The extremely low thickness profile permits the fabrication of the multiple stacked individual cells 20 in a small volume consistent with the volume available to receive an electrolytic storage capacitor within a conventional implantable device. As shown by FIG. 3, plural individual cells 20 can be easily arranged in a stack formation in which the anode current collectors 38 are abutting and therefore in electrical contact with each other to form a common anode terminal, and wherein the cathode current collector caps 33 are wired so that they are also electrically interconnected to form a common cathode terminal, thereby creating a battery module 16 (see FIG. 2) in which the battery cells 20 are permanently connected in an electrically parallel arrangement.

As further shown in FIG. 3, the resultant stack of cells will comprise a repeating pattern of electrolyte and electrode layers, with each electrode comprising either a first electrode layer set that includes an sequence of adjacent anode and anode collector layers, or a second electrode layer set that includes a sequence of adjacent cathode and cathode collector layers. For example, the pattern formed by the cells 20, starting from the left-hand side of the cell combination and proceeding to the right, is A-E-C-E-A-E-C-A, where the letter “A” represents an anode layer set, the letter “E” represents an electrolyte layer, and the letter “C” represents a cathode layer set. Advantageously, no insulation layers are required anywhere within the cell stack of a single battery module 16, such that battery module thickness can be minimized.

In order to fabricate a useful battery system for a high-energy implantable device, it is necessary to combine multiple cells in both series and parallel configurations. The invention achieves this by hardwiring the individual cells 20 of each battery module 16 in a parallel configuration, and then selectively connecting two or more battery modules 16 to each other in either a parallel charge configuration or a serial discharge configuration. FIG. 4 illustrates a single battery module 16 combined with associated switching circuitry 18 (as per FIG. 2) to provide a high-energy battery system subassembly 50. In the battery system subassembly 50, the battery module 16 is constructed (by way of example only) to have six parallel-connected battery cells 20, and the switching circuitry 18 is provided by a MOSFET switch 52 (or other suitable switching device) and an associated switch driver unit 54 of conventional design. Two terminals 56 labeled “Discharge +” and Discharge −” provide a discharge path when the switch 52 is in conduction. Isolation diodes 58 prevent the reverse flow of cell energy through a pair of charging terminals 59 labeled “Charge +” and “Charge −.”

The operation of the individual components shown in FIG. 4 is made clear in FIG. 5, which shows three interconnected battery system subassemblies 50 collectively providing a high-energy, high-voltage battery system 60. When charging of the individual cells 20 is required, a d.c. voltage of sufficient amount is applied to the “Charge +” and “Charge −” inputs 62. By way of example, if the individual cells 20 of the battery modules 16 are to be charged to 4.2 volts dc, the applied voltage should be higher by the amount necessary to forward bias the isolation diodes 58. If the forward voltage drop for each diode is 0.6 volts the charging voltage should therefore be on the order of 5.4 volts d.c. The isolation diodes 58 will be reverse biased when the charging voltage is removed.

When the battery system 60 is required to deliver high-voltage energy, a trigger pulse is applied by conventional timing circuitry (not shown) to the inputs 64 labeled “Discharge Trigger.” This signal is applied to the switch driver unit 54 of each battery system subassembly 50. Each switch driver unit 54 has the principal function of providing galvanic isolation between each of the interconnected battery modules 16, since they will be electrically connected in series during the discharge pulse. The switch driver units 54 each produce a voltage output pulse that is applied between the gate and source of its associated switch 52. This voltage output pulse causes each switch 52 to simultaneously conduct, resulting in a series connection of the battery cells 20 in each of the interconnected battery modules 16. The series connection will produce an output voltage on the “HV Out +” and “HV Out −” outputs 66 that is the sum of the individual battery module voltages. In this example using a single cell voltage of 4.2 volts dc, the resulting system output voltage pulse will be 12.6 volts dc. During the discharge period when the switches 52 are conducting, the positive circuit of the topmost battery module 16 in FIG. 5 and the negative circuit of the bottommost battery module 16 in FIG. 5 will be driven to the maximum output voltage difference of the entire assembly. The isolation diodes 58 of each battery system subassembly 50 will prevent the reverse flow of energy through the “Charge +” and “Charge −” inputs at 62 at this time. It should be understood that this concept of interconnected battery system subsystems 50 is not limited to three as shown in FIG. 5. Indeed, in order to provide the high-energy necessary for defibrillation or cardioversion, a configuration is taught below wherein 158 such subsystems are interconnected as shown.

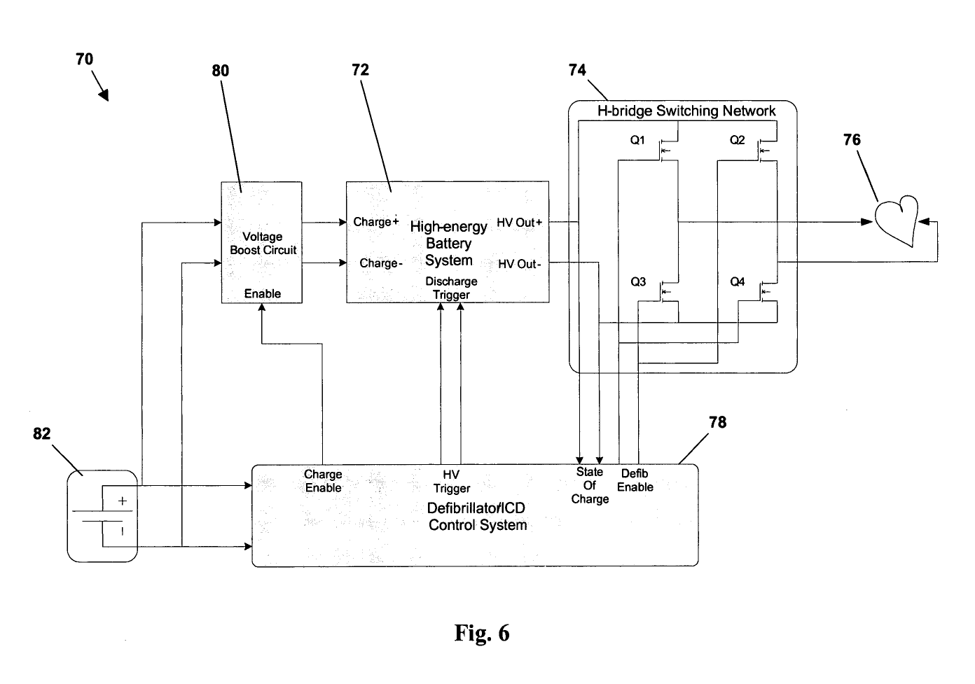

Turning now to FIG. 6, a first exemplary circuit arrangement 70 is shown that uses the battery system 60 of FIG. 5. The circuit 70 includes a high-voltage, high-energy battery system 72 (built with the battery system 60) whose high-voltage outputs are coupled to a conventional H-bridge switching network 74. The switching network 74 has four MOSFET transistors Q1, Q2, Q3 and Q4 wired in a cross-coupled configuration so that they are enabled in pairs, e.g. Q1/Q4 or Q2/Q3. The two outputs of the switching network 74 are connected by means of endocardial or epicardial electrodes (not shown) to a stimulus location on a heart 76, such as a ventricular or atrial wall thereof. Monitoring of the heart 76 and functional control of the circuit 70 is provided by a control system 78 that is conventionally implemented with a low-power microprocessor that would be familiar to those skilled in the art of implantable defibrillator/ICD design. Prime power for operation of the control system 78 in the circuit 70 is provided by a primary battery 82.

Under conditions of normal heart rhythm the battery system 72 is dormant and no signals are applied by the control system 78 to the inputs labeled “Discharge Trigger.” In the event that a condition such as tachycardia or fibrillation occurs in the heart 74, the condition will be sensed by the control system 78 by means of the electrodes and conventional sensing circuitry in the control system (not shown). If the condition exceed thresholds established within the control system 78, indicating a need for defibrillation or cardioversion, the control system 78 will assert its outputs labeled “HV Trigger” to cause the battery system 72 to provide high voltage at its outputs labeled “HV Out +” and “HV Out −.” The control system 78 will then assert its outputs labeled “Defib Enable” in an alternating sequence to cause the transistors Q1-Q4 within the switching network 74 to conduct. The transistors Q1-Q4 will conduct the high-voltage energy from the battery system 72 to the heart. By alternating the conduction of the transistor pairs Q1/Q4 and Q2/Q3 in the switching network 74, the circuit 70 device will deliver a bi-phasic defibrillation shock to the Heart 76. Upon completion of the defibrillation sequence, the control system 78 will negate its “HV Trigger” signals to the battery system 71.

The high-voltage outputs from the battery system 72 are also provided to the “State of Charge” inputs of the control system 78 for the purpose of monitoring the energy delivered to the heart and the state of charge of the battery system 72. In the event that the monitored voltage falls below a pre-determined threshold for the battery system 72, the control system 78 will assert its output labeled “Charge Enable.” This signal is connected to an optional voltage boost circuit 80 that is powered from a primary battery cell 82. The voltage boost circuit 80 is conventionally adapted to convert the energy from the primary cell 82 to the voltage required to charge the cells of the battery system 72, assuming these voltages are different.

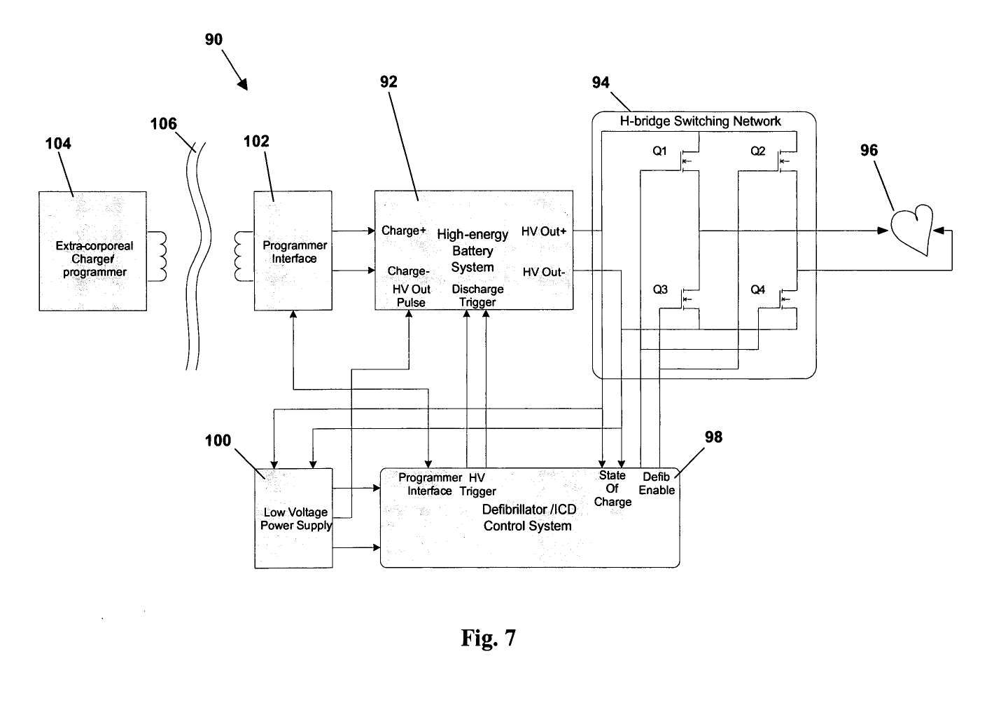

Turning now to FIG. 7, a second exemplary circuit arrangement 90 is shown that uses the battery system 60 of FIG. 5. Like the circuit 70, the circuit 90 includes a battery system 92 (built with the battery system 60), an H-bridge switching network 94 for delivering electrical impulses through a lead system to a heart 96, and a control system 98. Unlike the circuit 70, the circuit 90 does not include a primary battery or voltage boost circuit, and instead comprises a low-voltage power supply 100 and a programmer interface 102. In the circuit 90, the circuit operation with respect to patient therapy is identical to that described for FIG. 6. Under direction of the control system 98, the battery system 92 provides high-voltage current to the switching network 94 in order to deliver energy to the Heart 96. Insofar as there is no primary battery, prime power for the control system 98 is provided from the battery system 92 via the power supply 100. Note that the energy requirements for the control system 98 are miniscule, perhaps 60 microwatts continuously. The power supply 100 can be implemented with a charge-pump or similar topology (not shown) wherein short pulses of high-voltage energy are periodically applied to an energy storage capacitor (not shown) to maintain a constant lower voltage for powering the control system 98. The power supply 92 will also periodically assert a signal on its output line connected to the input of the battery system 92 labeled “HV Out Pulse.” Assertion of this signal will cause the battery system 92 to momentarily produce output voltage from its “HV Out +” and “HV Out −” outputs in order to transfer energy to the power supply 100.

Using the thin-film battery technology disclosed herein, the battery system 92 should be easily capable of storing enough energy to operate the control system 98 for over one year and also deliver some number of defibrillation/cardioversion pulses. The battery system 92 can be periodically recharged by energy supplied from an extra-corporeal charger/programmer 104 through the patient skin 106. The charger/programmer 104 generates an a.c. electromagnetic field which is inductively coupled to the programmer interface 102 to transfer energy to the battery system 92.

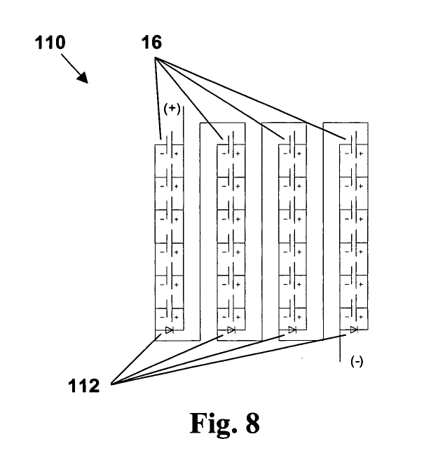

Turning now to FIG. 8, a high-energy, high-voltage battery system 110 is shown in which four battery modules 16 (or any other desired number) are permanently connected in series while eliminating the isolation (steering) diodes 58 and switching network 18 shown in FIG. 4. Assuming each module 16 has a nominal operating voltage of 3.7 volts, the output voltage for the battery system 110 will be 14.8 volts d.c. External electrical connections are provided at the positive (+) and negative (−) ends of the interconnected modules. Each battery module 16 may be equipped with a blocking diode 112 that prevents reverse polarization of the battery module. These diodes also allow the battery system 110 to provide energy at reduced voltage in the event that any single battery modules fails in an open circuit state.

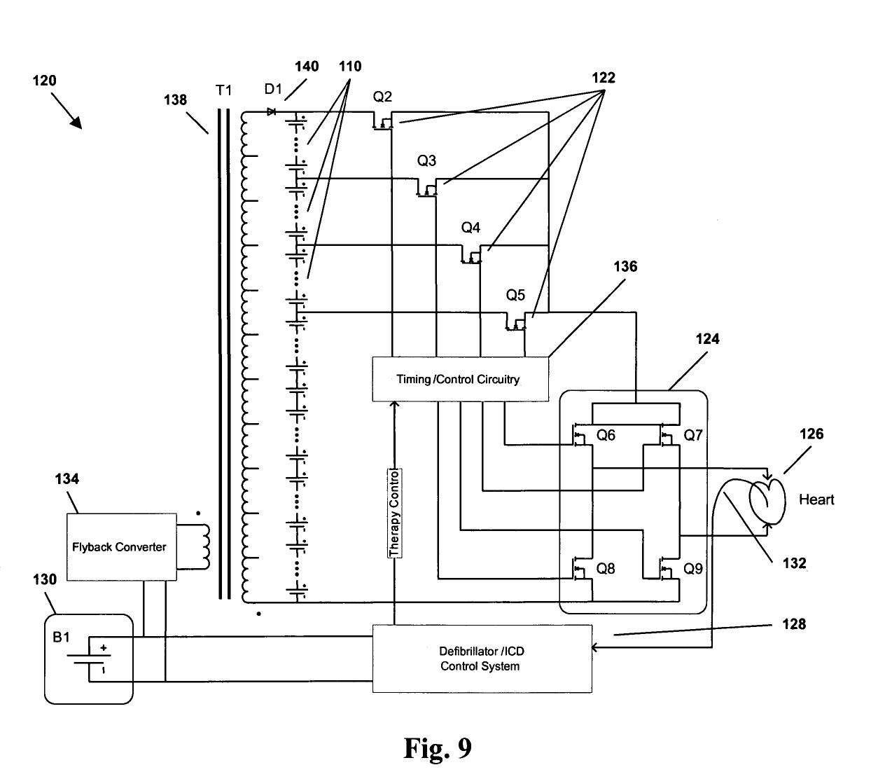

Turning now to FIG. 9, an exemplary circuit arrangement 120 is shown that uses the battery system 110 of FIG. 8. The circuit 120 includes one or more battery systems 110 (depending on the voltage output of each such system and the total desired voltage), a set of (e.g., four) voltage selection switching transistors 122 (Q2-Q5), an H-bridge switching network 124 for delivering electrical impulses through a lead system to a heart 126, a defibrillator/ICD control system 128 and a primary battery 130 (B1). An endocardial catheter 132 contains the leads to the heart 126 and is also connected to the control system 128 as an input to provide electrical signals related to cardiac activity. Unlike the previously described circuit arrangements of FIGS. 6 and 7, the circuit 120 also includes a flyback converter circuit 134.

In the circuit 120, the circuit operation with respect to patient therapy is identical to that described for FIGS. 6 and 7. The control system 128 continuously monitors cardiac activity via the catheter 132. In the event of abnormal cardiac activity requiring high-energy therapy, the control system 128 provides output signals to timing/control circuitry 136. The timing/control circuitry 136 has as its outputs the gates of the voltage selection transistors 122 and the transistors (Q6-Q9) of the H-bridge network 124. The voltage selection transistors 122 are connected to tap into the battery system(s) 110 at different voltage reference points of the series-connected battery modules 16 therein. It will be seen that transistor Q2 taps in at the highest voltage reference point of the battery system(s) 110 whereas the remaining transistors Q3, Q4 and Q5 tap in at successively lower voltages. The tap voltages are selected according to the therapy output requirements of the circuit 120, as are the number of transistors and corresponding voltage tap points.

In order to deliver high energy therapy to the heart, the timing/control circuitry 136 will energize the gate of only one of the voltage selection transistors 122 to connect the positive input of the H-bridge switching network 124 to one of the voltage tap outputs of the battery system(s) 110. Two of the transistors (e.g. Q6 and Q9) in the H-bridge network 124 will then be energized to deliver energy to the heart 132. The magnitude of the voltage delivered to the H-bridge network 124 and subsequently delivered as therapy to the heart 132 is determined by which of the voltage selection transistors 122 is energized during the operation of the H-bridge network. In the event that a bi-phasic pulse is to be delivered, the timing/control circuitry 136 will de-energize the two transistors first energized in the H-bridge network 124 and then energize the opposing transistors, e.g. Q7 and Q8. If the therapy regimen requires the second pulse of the biphasic pair to be of equal amplitude (but opposite polarity) then the timing/control circuitry 136 will maintain the state of the voltage selection transistor 122 first energized. In the event that the therapy regimen requires a different amplitude for the second pulse of the biphasic pair, the timing/control circuitry 136 will de-energize the active voltage selection transistor 122 and energize a voltage selection transistor connected to one of the other voltage taps on the battery stack before energizing the second pair of transistors in the H-bridge network 124. It should be understood that this configuration also supports the delivery of multiple monophasic pulses of equal or varied amplitude as well as bi-phasic pulse of equal or varied amplitude.

After delivery of a number of defibrillation pulses, the battery system(s) 110 may require recharging. The flyback converter 134, which is of conventional design, is provided for this purpose. The primary battery 130 provides energy to the flyback converter 134, which is enabled by the control system 128. When the flyback converter 134 is enabled, energy is delivered to the magnetic core of a transformer 138 (T1) and subsequently released to the secondary winding. The primary:secondary turns ratio of the transformer 138 is established to provide a significant increase (step-up) in voltage so that a charging voltage commensurate with the operating voltage of the battery system(s) 110 is delivered. A diode 140 conducts the energy released from the transformer 138 into the battery system(s) 110 and prevents the reverse flow of current between switching cycles or when the flyback converter 134 is disabled.

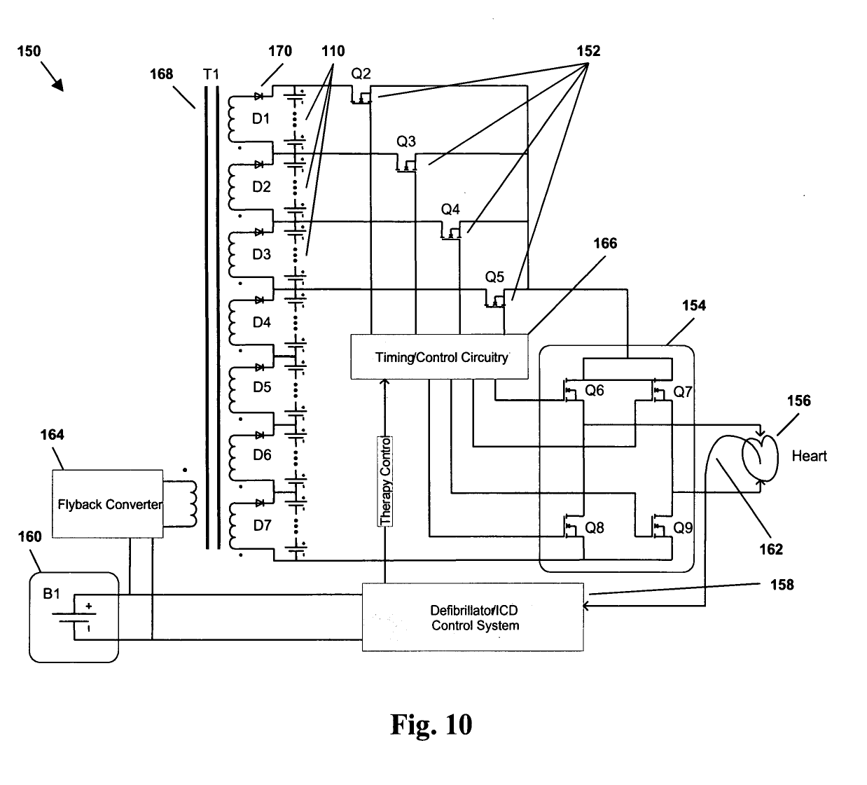

Turning now to FIG. 10, a second exemplary circuit arrangement 150 is shown that uses the battery system 110 of FIG. 8. The operation of the circuit 150 with respect to delivery of energy from the battery system(s) 110 to the heart 156 is identical to that described for FIG. 9. Thus, the voltage selection transistors 152, the H-bridge switching network 154, defibrillator/ICD control system 158, the primary battery 160, the endocardial lead 162, the flyback converter 164, and the timing/control circuitry 166 operate identically to their counterparts in FIG. 9. What is different is the technique used for recharging the battery system(s) 110 with respect to the flyback transformer 168. In the circuit 150, the transformer 168 is equipped with multiple secondary winding sets, each of which is connected to a rectifier diode 170. Each secondary winding set and rectifier diode 170 is connected to a segment of the battery system(s) 110 (representing some number of the modules 16) so that all segments of the battery system(s) are recharged when the flyback converter 164 is energized.

Comparing the recharging configurations of FIGS. 9 and 10, the recharging configuration depicted in FIG. 9 requires the simplest component arrangement, namely, a single secondary winding on the transformer 138 and a single blocking diode 140. However, in order to ensure that the recharging energy is equitably distributed between the individual segments of the battery system(s) 110, all segments must be reasonably matched in impedance and capacity. The recharging configuration of FIG. 10 has additional complexity, including multiple taps on the secondary winding of transformer 168 and multiple blocking diodes 170. However, this additional complexity provides the benefit of regulating the recharging of the battery system(s) 110 to ensure that the recharging energy is equitably distributed even when the individual segments of the battery system(s) 110 are not ideally matched in impedance and capacity.

Rationale for Configuration

Most commercially available implantable defibrillators and ICDs are capable of producing defibrillation shocks at a peak voltage of about 600 volts and a total energy of about 30 joules, substantially all of which is delivered within about 20 milliseconds to the tissue being stimulated. This energy is delivered through endocardial electrodes with a typical impedance of 40 ohms. The peak current required at this voltage and impedance is:
V/R=I; 600 volts/40 ohms=15 amperes
Each of the above-described battery modules 16 can be designed to support this current level during the defibrillation pulse.

The battery cells 20 shown in FIG. 4 are reported in the '968 patent to produce a continuous discharge current density of 82.4 mA-cm⁻². At this level, the total electrode surface area required for each battery module 16 is:

ti 15 A/0.0824 A-cm⁻²=182 cm²

In the device 2 of FIG. 1, the available surface area for a single cell in the stacks 10A and 10B was said to be 2 cm*6 cm=12 cm². A battery module 16 would require the following number of parallel-connected cells 20 to support the required discharge current:
182 cm²/12 cm²-cell⁻¹=15.17 cells=>15 parallel cells

Each battery cell 20 shown in FIG. 4 has a thickness of 14 μm. A battery module 16 of seventeen parallel-connected cells each having a thickness of 14 μm per cell will have a resulting thickness of:
(15 parallel cells*14*10⁻⁶ m-cell⁻¹=0.210 millimeters

The operating voltage for a representative cell 20 varies over the range of 4.2 volts at full charge to 3.4 volts when fully discharged. If the mean voltage is take to be 3.8 volts under load during discharge, the total number of battery modules 16 required to deliver the required 600 volts, and the total cell stack thickness, is:
600 volts/3.8 volts-cell subsystems⁻¹=157.89=>158 battery modules
158 battery modules*0.210 millimeters=33.18 millimeters=3.32 cm

In the device 2 of FIG. 1, there are two cell stacks 10A and 10B. If the required cell stack thickness is evenly divided between the stacks, each cell stack 10A and 10B will each require 1.66 cm, not including a stack substrate, if such is used. According to the '424 patent publication, a polyimide substrate that can be used in a thin-film battery will range in thickness between 25-75 μm. Moreover, a thin layer of insulative material, such as parylene, will be required between each battery module 16 for insulation purposes. Assuming a 1 μm insulation layer is disposed between each battery module 16, and because there will be 79 battery modules in each cell stack 10A and 10B, there will be 79−1=78 1 μm thick insulation layers per stack, and 78 μm of thickness must be additionally added. The total thickness of each cell stack 10A and 10B will thus be 1.66 cm+78 μm+30 μm=>1.67 cm. This is within the 1.7 cm interior height specified for the component cavity 4 of the device 2. The volume of each cell stack is:
2 cm*6 cm*1.67 cm=20.04 cm³
This is comparable to the volume required for aluminum electrolytic storage capacitors as presently used in defibrillators and ICDs.

According to the '968 patent, the energy capacity of each battery cell 20 is 7.2 watt-seconds (joules)-cm⁻². For an individual cell electrode surface area of 12 cm² and 15 cells in parallel combination, the total energy capacity for a battery module 16 is:
15 cells*12 cm²-cell⁻¹*7.2 j-cm⁻²=1296 j
Each battery module 16 will therefore have the capacity to deliver at least 43 defibrillation shocks of 30 joules each before requiring recharging.

The application of lithium secondary cells to implantable medical applications has been limited to date by poor cell performance with respect to cycle life, energy density and self-discharge. The use of thin-film cells in implantable devices is proposed by John Bates and Nancy Dudney in “Thin Film Rechargeable Lithium Batteries for Implantable Devices.” ASAIO Journal 1997; 43:M644-M647. The authors present data that predicts significant improvement in rechargeable cell cycle life and energy density. Similar improvements are disclosed in the '968 patent.

Another benefit of the thin-film technology is significant reduction in cell self-discharge as a result of improved electrolyte performance over traditional liquid or polymer electrolyte cell designs. In tests conducted by Nancy Dudney, et al. at Oak Ridge National Laboratories, very small capacity cells were constructed with constituent components disclosed in U.S. Pat. No. 5,569,520 of Bates (referenced above). After fabrication, the cells were stored and periodically monitored to assess self-discharge by measuring the cell terminal voltage. The data predicts a relationship wherein self-discharge is directly proportional to the electrode surface area and inversely proportional to the electrolyte layer thickness. This leads to a self-discharge rate of 0.6 μAh-cm⁻²-year⁻¹ with an electrolyte layer thickness of 1.2 μm. When this predicted rate is applied to a 15-cell battery module, the predicted self discharge rate is:
0.6 μAh-cm⁻²-year⁻¹*12 cm²*15 cells=108 μAh-year⁻¹
The battery module 16 has a capacity of 1483 mAh when configured with 15 cells, so the rate of self-discharge expressed as a percentage is:
(45 μAh-year⁻¹/1483 mAh)*100=0.03%-years⁻¹
This low rate of self-discharge enables the application of these cells to implantable systems without sacrificing device lifetime due to wasted energy.

In the circuit 90 of FIG. 7, the battery system 92 is used to provide energy for the low-voltage background loads of the implantable device. By way of example, a representative device might require 2.8 volts d.c. at 30 μA for monitoring and pacing loads. The total energy requirement for one year of operation would be:
2.8 VDC*30 μA*31.56*10⁶ sec-year⁻¹=2651 watt-second-year⁻¹
If the efficiency of the voltage step-down process is estimated at 75% and the patient requires no more than two defibrillations, the battery would be capable of supporting all device operation for at least 60 weeks. This embodiment therefore eliminates the need for a primary battery by stipulating that the high-voltage secondary battery be recharged periodically, perhaps every 12 months.

The alternate embodiments disclosed in FIGS. 9 and 10 provide the capability to deliver varied electrical stimulation therapy not available in capacitive discharge defibrillation systems as currently practiced. Specifically, the circuits disclosed in FIGS. 9 and 10 provide the capability to deliver electrical energy wherein the peak voltage of each and every discharge pulse is selectable and independent of every other pulse. The present practice for implantable defibrillators and ICDs is to utilize one or more high voltage energy storage capacitors to deliver the defibrillation energy in a single monophasic or biphasic pulse. Because the tissue to be stimulated presents an electrical load that is primarily resistive, the voltage waveform of the resulting discharge is fundamentally limited to a decaying exponential shape. In the case of a biphasic pulse, the discharge pulse is typically interrupted when the capacitor voltage has decayed 50%-60% from its initial value. The capacitor connection is then electronically reversed within the device to deliver the remaining stored energy with voltage polarity opposite that of the initial pulse. The biphasic capacitive discharge pulse has been clinically proven to be more efficacious for defibrillation than monophasic pulses of equivalent energy and is therefore chosen today for the vast majority of patients receiving ICDs.

There are indications that defibrillation efficiency may be further improved by the use of a modified capacitive discharge system or by the application of voltage discharge waveforms that are not exponential. In the case of the former, animal studies have been conducted to determine the effect on biphasic defibrillation energy thresholds as the voltage change at the phase reversal is varied. The results of one group of studies indicate that the defibrillation threshold in pigs could be improved by increasing the leading edge voltage of the second phase of a biphasic pulse. See Yamanouchi et al, “Large Change in Voltage at Phase Reversal Improves Biphasic Defibrillation Thresholds” Circulation 1996;94: 1768-1773. With respect to waveforms that are not exponential, two studies with guinea pigs suggest that defibrillation efficacy is strongly affected by the overall waveform and that one optimal waveform may exist. These tests are discussed in Malkin, R. “Large Sample Test of Defibrillation Waveform Sensitivity” Journal of Cardiovascular Electrophysiology, 2002;13:361-370 and Guan et al. “Analysis of the Defibrillation Efficacy for 5-ms Waveforms” Journal of Cardiovascular Electrophysiology 2004; 15:447-454.

The alternate embodiments disclosed in FIGS. 9 and 10 also provide a battery circuit configuration that is much less complex than prior art such as the '351 patent of Adams. Whereas this reference teaches the use of multiple switches to affect either series or parallel connections of the batteries for discharging and charging, the circuits of FIGS. 9 and 10 permit the charging and discharging of multiple batteries in a simple permanent series connection. This simpler configuration reduces the number of components required to implement a practical high-voltage high-energy battery system.

Accordingly, a high-energy battery power source for implantable medical use has been disclosed. Although specific exemplary embodiments have been shown and described, it will be apparent that various modifications, combinations and changes can be made to the disclosed designs in accordance with the invention. It should be understood, therefore, that the invention is not to be in any way limited except in accordance with the spirit of the appended claims and their equivalents.

What is claimed is:

1. A high-energy battery power source for implantable use, comprising:
an input;
an output;
two or more battery modules;
each battery module comprising two or more rechargeable battery cells;
said battery cells being of relatively low voltage and permanently configured within each battery module in an electrically parallel arrangement;
said battery modules being permanently connected to each other in series;
a low-voltage primary power source; and
a high-voltage charging system powered by said primary power source for charging said series-connected battery modules.

2. A power source according to claim 1, wherein said relatively low voltage is approximately 3.4-4.2 volts and said relatively high voltage is approximately 600 volts.

3. A power source according to claim 1, wherein each of said battery modules produces peak current at a discharge level of approximately 15 amperes.

4. A power source according to claim 1 wherein said implantable device is one of an implantable defibrillator or an implantable cardioverter-defibrillator.

5. A power source according to claim 1 wherein said battery cells comprise large surface area, thin-film structures and wherein the battery cells of each of said battery modules are arranged in a stack.

6. A power source according to claim 5 wherein said battery modules are arranged in one or more stacks.

7. A power source according to claim 1 wherein said primary power source comprises a battery.

8. A power source according to claim 1 wherein said charging system comprises a flyback transformer.

9. A power source according to claim 1 wherein said charging system comprises a flyback transformer having plural secondary winding sets each connected to a segment of said series-connected battery modules.

10. A power source according to claim 1 further including a plurality of voltage reference taps on said series-connected battery modules and a control system adapted to selectively activate said voltage taps to provide a therapy regimen utilizing controlled voltage pulses at different voltage levels.

11. An implantable device for delivery of high-energy electrical stimulus to living tissue, comprising:
a case;
a connector block on said case for attachment of implantable leads;
a component cavity within said case;
a high-energy battery power source disposed in said component cavity, comprising:
an input;
an output;
a stack of battery modules;
each battery module comprising a stack of battery cells;
said battery cells being of relatively low voltage and permanently configured within each battery module in an electrically parallel arrangement;
said battery modules being permanently connected to each other in series;
a low-voltage primary power source; and
a high-voltage charging system powered by said primary power source for charging said series-connected battery modules.

12. An implantable device according to claim 11, wherein said relatively low voltage is approximately 3.4-4.2 volts and said relatively high voltage is approximately 600 volts.

13. An implantable device according to claim 11, wherein said peak current discharge level is approximately 15 amperes.

14. An implantable device according to claim 11 wherein said implantable device is one of an implantable defibrillator or an implantable cardioverter-defibrillator.

15. An implantable device according to claim 11 wherein said battery cells comprise large surface area, thin-film structures and wherein the battery cells of each of said battery modules are arranged in a single stack.

16. An implantable device according to claim 15 wherein said battery modules are arranged in a pair of stacks.

17. An implantable device according to claim 11 wherein said primary power source comprises a battery.

18. An implantable device according to claim 11 wherein said charging system comprises a flyback transformer.

19. An implantable device according to claim 11 wherein said charging system comprises a flyback transformer having plural secondary winding sets each connected to a segment of said series-connected battery modules.

20. An implantable device according to claim 11 further including a plurality of voltage reference taps on said series-connected battery modules and a control system adapted to selectively activate said voltage taps to provide a therapy regimen utilizing controlled voltage pulses at different voltage levels.

21. A high-energy, thin-film battery cell stack power source unit for an implantable medical device, comprising:
a stacked sequence of battery modules;
each battery module comprising a stacked sequence of large surface area, thin-film battery cells of relatively low voltage;
said stacked sequence of battery cells in a battery module comprising a repeating pattern of electrolyte and electrode layers and being substantially free of insulation layers;
said electrode layers including anode layer sets that are permanently electrically connected to each other to define an anode terminal of a battery module, and cathode layer sets that are permanently electrically connected to each other to define a cathode terminal of said battery module, such that the battery cells of said battery module are connected in an electrically parallel arrangement;
said battery modules being permanently connected to each other in series;
a low-voltage primary power source; and
a high-voltage charging system powered by said primary power source for charging said series-connected battery modules.

22. An implantable device for delivery of high-energy electrical stimulus to living tissue, comprising:
a case;
a connector block on said case for attachment of implantable leads; a component cavity within said case;
a high-energy, thin-film battery cell stack power source unit, comprising:
a stacked sequence of battery modules;
each battery module comprising a stacked sequence of large surface area, thin-film battery cells of relatively low voltage;
said stacked sequence of battery cells in a battery module comprising a repeating pattern of electrolyte and electrode layers and being substantially free of insulation layers;
said electrode layers including anode layer sets that are permanently electrically connected to each other to define an anode terminal of a battery module, and cathode layer sets that are permanently electrically connected to each other to define a cathode terminal of said battery module, such that the battery cells of said battery module are connected in an electrically parallel arrangement;
said battery modules being permanently connected to each other in series;
a low-voltage primary power source; and
a high-voltage charging system powered by said primary power source for charging said series-connected battery modules.

23. A method of use for an implantable device for delivery of high-energy electrical stimulus to living tissue, the device comprising:
a case;
a connector block on said case for attachment of implantable leads;
a component cavity within said case;
a high-energy battery power source disposed in said component cavity, comprising:
an input;
an output;
a stack of battery modules;
each battery module comprising a stack of battery cells;
said battery cells being of relatively low voltage and permanently configured within each battery module in an electrically parallel arrangement;
said battery modules being connectable to each other in series;
a plurality of voltage taps on said battery modules; and
a control system adapted to selectively activate said voltage taps to provide a therapy regimen utilizing pulses at different voltage levels;
said method comprising delivering a sequence of bi-phasic and/or monophasic pulses each having a controlled waveform resulting from said control system selectively activating said voltage taps and controlling pulse duration.
